(19) United States
(12) Patent Application Publication
(10) Pub. No.: US 2005/0071304 A1
(43) Pub. Date: Mar. 31, 2005

(54) Apparatus for the classification of physiological events

(75) Inventor: Richard A. Schomburg, Hillsboro, OR (US)

Correspondence Address: HAHN LOESER & PARKS, LLP, One GOJO Plaza, Suite 300, AKRON, OH, 44311-1076, US

(73) Assignee: Biotronik Mess-und Therapiegeraete GmbH & Co.

(21) Appl. No.: 10/674,280

(22) Filed: Sep. 29, 2003

(51) Int. Cl. A61B 5/00; G06F 15/18; G06G 7/00; G06E 3/00; G06E 1/00

(52) U.S. Cl. 706/20; 600/300

(57) ABSTRACT

An apparatus according to the invention for the classification of physiological events on the basis of physiological signals which represent or constitute the physiological events by means of a probabilistic neural network 5 includes a probabilistic neural network 5 which is adapted to receive a set of values representing the physiological signal and which contains a number of event classes which represent physiological events and which are respectively determined by a number of comparative values, which network is adapted on the basis of the comparison of the set of values with the comparative values to implement an association of the physiological signal represented by the set of values with one of the event classes, and an updating unit 10 connected to the probabilistic neural network 5 for updating the comparative values of an event class on the basis of the set of values of at least one physiological signal which has been associated with said event class in a preceding association operation.

The present invention concerns an apparatus for the classification of physiological events, in particular physiological events such as for example cardiac reactions on the basis of an electrocardiogram.

### BACKGROUND OF THE ART

Physiological events give rise to physiological signals or themselves represent signals, on the basis of which they can be classified. The classification of physiological events or signals is useful in particular in relation to implantable medical devices such as for example cardiac pacemakers or implantable defibrillators in order to distinguish events requiring treatment from those which are not in need of treatment, or events in respect of which different treatments are indicated. On the basis of the classification procedure the implantable medical device is put into the position of automatically triggering off the treatment which is possibly required.

Previous apparatuses for the classification of physiological events, in particular intracardial events, in implantable medical devices are essentially based on filtering of the signal shape and on the provision of a threshold value or a plurality of threshold values in combination with time analysis in respect of the value exceeding/falling below the threshold value or values.

In order to achieve acceptable sensitivity to the signals of physiological events and acceptable distinguishability of events with the known apparatuses, it is necessary, during the cardiac cycle in which an event occurs, to suspend the recording of further physiological signals. However such suspension excludes the reliable detection of various important classes of intracardial events and the effective treatment thereof, thus for example an abnormal relationship between the two chambers of the heart.

Therefore the object of the present invention is to provide an improved apparatus for the classification of physiological events, in particular intracardial events, which helps to overcome the above specified disadvantages.

### SUMMARY OF THE INVENTION

That object is attained by an apparatus for the classification of physiological events as set forth in the accompanying claims. The appendant claims set forth advantageous configurations of the invention.

An apparatus according to the invention for the classification of physiological events on the basis of physiological signals displaying or representing the physiological events by means of a probabilistic neural network includes:
a probabilistic neural network which is adapted to receive a set of values representing the physiological signal and which contains a number of event classes which represent physiological events and which are respectively determined by a number of comparative values, which network is adapted on the basis of the comparison of the set of values with the comparative values to implement an association of the physiological signal represented by the set of values with one of the event classes, and
an updating unit connected to the probabilistic neural network for updating the comparative values of an event class on the basis of the set of values of at least one physiological signal which has been associated with said event class in a preceding association operation.

In this respect a physiological signal which prior to input into the classification unit has been prepared, for example standardized, filtered, adjusted etc., is also to be considered as the physiological signal. The physiological signal can itself be viewed as the physiological event or can be caused by the physiological event.

The present invention which is suitable in particular for use in an implantable medical device, for example a cardiac pacemaker or defibrillator, is based on the following realizations:

Probabilistic neural networks are suitable for the classification of physiological events on the basis of physiological signals representing them. In such a probabilistic neural network, classification is effected on the basis of a comparison of a set of coefficients as a set of values which represent the physiological signal, with a set of comparative coefficients as comparative values which represent the signal shape which is typical for a given physiological event, that is to say for the event class of the signal. The signal or the event on which the signal is based is then associated with that event class in respect of which there is the greatest degree of similarity between the set of coefficients and the comparative coefficients.

A set of comparative coefficients is associated with a respective node in the probabilistic neural network. A plurality of sets of comparative coefficients can also be associated with an event class, in which case then each of those sets is associated with its own node so that there are a number of nodes (a node cluster) for that event class.

Hitherto the comparative coefficients for an event were established in such a way that to begin with the coefficients of signals with the signal shape typical in respect of the respective event class were ascertained and associated with a node as comparative coefficients. In the case of implantable medical devices the operation of establishing the comparative coefficients can be effected for example during or shortly after the implantation procedure.

The described way of establishing the comparative coefficients however involves the consequence that the comparative coefficients are established for the future. If the typical signal shape of signals which represent physiological events or which go back thereto gradually alters with the passage of time, the result of this can be that the comparative coefficients of the corresponding event class no longer adequately describe the associated signal shape.

There are event classes for which absolutely established comparative coefficients are not necessary or in respect of which the change in the signal shape represents a significant manner of behavior. For those event classes, the coefficients which were established to start with can therefore result in an inaccuracy in classification, which makes it difficult or even impossible to effectively distinguish between signals which belong to a corresponding event class and those which do not belong thereto. In order to avoid the reduction in the level of classification accuracy for event classes of that nature, the classification apparatus according to the invention therefore includes an updating unit, by means of which the comparative coefficients are updated and are thus adapted to gradual changes in the typical signal shapes of signals, representing physiological events, of the corresponding event classes. In that way it is possible to durably maintain an effective distinction between signals which belong to an event class and those which do not belong thereto.

In a configuration of the updating unit it is designed in such a way that, upon updating of the comparative values, an average value is formed from a number of value sets which have previously resulted in an association of the physiological signals which they represent with the event class to be updated. The apparatus therefore preferably includes an averaging unit for value sets. The updating operation is then effected on the basis of the average value formed in that way. By virtue of the formation of the average value, it is possible to prevent updating of an event class solely on the basis of a signal which admittedly still belongs to the corresponding event class but which in comparison with other signals associated with that class is at the edge of the region of the associated signal shapes. Classification on the basis of a signal of that kind could have the result that the updated comparative values do not adequately reflect the actual change in the signal shape which is typical for the event class.

In an alternative configuration of the updating unit it is designed in such a way that upon updating of the comparative values exponential weighting of a number of value sets which previously resulted in an association of the corresponding physiological signals with the event class to be updated is effected. The apparatus thus includes an evaluation unit for value sets respectively characterizing a detected physiological signal. Updating is then effected on the basis of the exponentially weighted value sets. In this embodiment it is possible to take account of all value sets associated in the past in the event class.

Irrespective of the design configuration of the updating unit updating of an event class can be effected after the association of the n-th value set with that event class. In that respect n can also be of the value one, that is to say updating is effected continuously with each value set which is associated with the corresponding event class. The choice of the value for n, that is to say the frequency of updating, can in that case be effected having regard to the long-term behavior of the event classes. Different values for n can also be involved for different event classes so that the frequency of updating can be particularly well adapted to different demands of various event classes.

In a development the apparatus according to the invention also includes:
a signal input for the input of a physiological signal; and
a transformation unit which is connected to the signal input for receiving the physiological signal and which is adapted to implement a transformation of the physiological signal in such a way that as the output signal it outputs a number of values representing the physiological signal and based on the transformation operation; wherein the probabilistic neural network is connected to the transformation unit for receiving the values as the value set.

In a preferred configuration of that development the transformation unit is adapted for executing the transformation operation on the basis of wavelets and a transformation rule determining the values to be outputted using the wavelets. Wavelet transformation is simple to implement and makes it possible to represent signals with relatively few values (in the form of coefficients). At the same time, in wavelet transformation, sufficient information about the signal is still retained to ensure reliable classification in the probabilistic neural network. In addition wavelet transformation affords the possibility of adapting the transformation operation, within the limits existing for calculation of the transformation procedure, to the effective recognition of individual event classes. Preferably, besides values which describe a stem wavelet, the values obtained by the wavelet transformation operation additionally include scaling values and transformation values which, in relation to a respective stem wavelet characterize the form of the input signal (physiological signal).

### BRIEF DESCRIPTION OF THE DRAWINGS

Further features, properties and advantages of the present invention will be apparent from the description hereinafter of an embodiment with reference to the accompanying drawings in which:

FIG. 1 shows an embodiment of the present invention, and

FIG. 2 shows the updating unit of the illustrated embodiment.

### DETAILED DESCRIPTION OF A PREFERRED EMBODIMENT

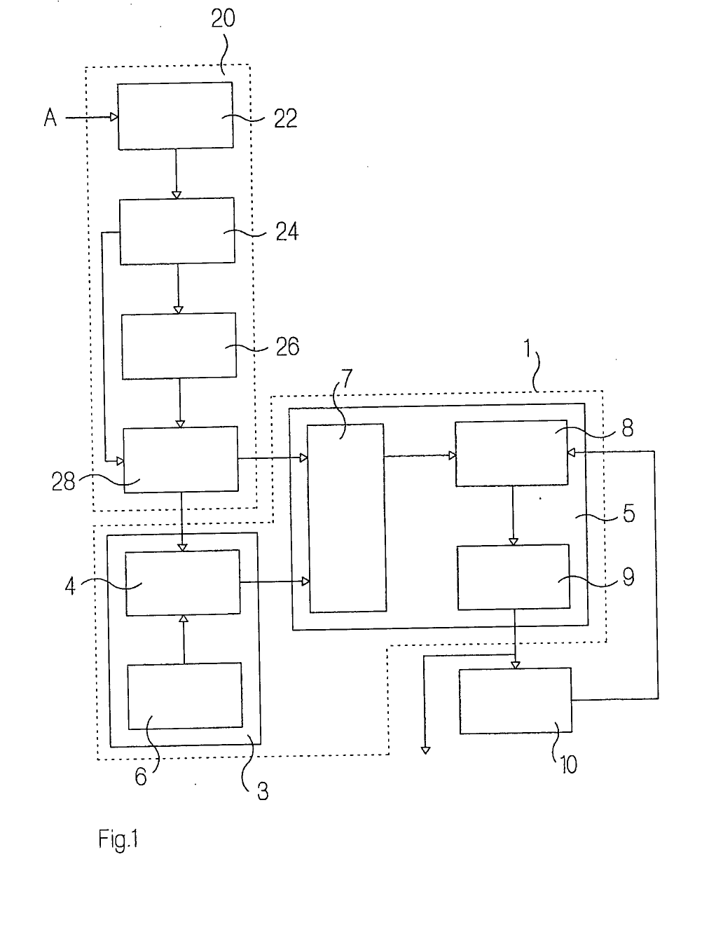

In the embodiment illustrated in FIG. 1 the apparatus for the classification of physiological events has a classification unit 1 which includes a transformation unit 3 and a probabilistic neural network 5 which is connected to the transformation unit 3 for receiving coefficients (that is to say, values) which represent a physiological signal which passes into the transformation unit 3 and is possibly processed therein. In the present embodiment, also connected upstream of the transformation unit 3 is a signal preparation unit 20 which includes an anti-aliasing filter 22, a broadband analog-digital converter 24, referred to hereinafter for the sake of brevity as the A/D-converter, a detection stage 26 for the detection of a physiological event and a combined adjusting/standardizing stage 28, an incoming physiological input signal A passing through those stages in that sequence, the stage 28 being connected to the transformation unit to output a processed physiological signal. In addition the apparatus for the classification of physiological events includes an updating unit 10 which is connected to the probabilistic neural network 5 for receiving the output signal thereof and for updating the probabilistic neural network 5 on the basis of the output signal.

The averaging unit mentioned in the introductory part of this specification, or the evaluation unit, are preferably component parts of the updating unit and are not further illustrated in FIG. 1.

Hereinafter, signal preparation, which is implemented in the signal preparation unit 20, of the physiological input signal A which in the present embodiment is an intracardial electrogram (IEGM), as is to be recorded for example by means of a cardiac pacemaker, will be briefly discussed. It should be pointed out however that the physiological signals which can be classified with the present invention are not limited to intracardial electrograms.

The anti-aliasing filter 22 involves filtering of the IEGM by means of an anti-aliasing low-pass filter as well as suitable amplification and/or scaling of the IEGM. As is known for sampled data systems, the filter suppresses signal components which can occur at frequencies above half the sampling rate and are superimposed by the subsequent signal processing steps. In addition no further filtering is effected to maintain the accuracy and the morphology of the signal shape of the IEGM.

The filtered IEGM is passed by the anti-aliasing filter 22 to the A/D-converter 24 which is a conventional analog-digital converter with a stepwise linear relationship between the input signal and the output signal. The sampling rate and the resolution of the output signal are adapted to the demands of the classification procedure. In general they are at 1024 Hz or below or at 8 bits or above, Depending on the requirements involved it is possible to use various A/D-converter architectures, including the so-called “one-bit design”. In special cases in which there are input signals with a large dynamic range, the use of nonlinear A/D-converter structures (which are companding, that is to say which compress the signal and then expand it again) may be advantageous. The converted IEGM is passed by the A/D-converter 24 as an output signal to the detection stage 26 and to the adjusting/standardizing stage 28.

The detection stage 26 involves the detection of an event on the basis of threshold consideration which is rate-adaptive from one event to another. The result of detection is indicated by an activity of the signal shape of the input signal. If the detection stage 26 detects an event it outputs a trigger signal (triggering signal) to the adjusting/standardizing stage 28 which triggers adjustment and/or standardization of the physiological signal.

If the adjusting/standardizing stage 28 receives a trigger signal from the detection stage 26, the underlying IEGM is detected in an event window with a predetermined window width which is generally 64 sampling steps, and centered in the window. The window is adapted to the expected type of event. The procedure also involves ascertaining the time interval from the last-detected event to the present event and standardization of the signal shape to a standardized peak-to-peak amplitude on the basis of a standardization factor in order to obtain a standardized event signal. The adjusting/standardizing stage 28 transmits the time interval and the standardization factor to the probabilistic neural network 5 whereas it transmits the event signal which is standardized and centered in the window to the transformation unit 3.

The transformation unit 3 executes wavelet transformation of the centered and standardized event signal, the result of the transformation operation being a number of coefficients representative of the signal. Wavelet transformation is a well-known method of compactly representing any signals. In that case, the transformation of a signal is effected by means of reference wavelets and a calculation procedure which specifies how the reference wavelets are to be calculated with the signal. Details of the transformation can be selected within the mathematical limits given by the calculation environment, in such a way that it can be highly effectively used for given signal classes. In the present embodiment which is intended for use in an implantable medical device, wavelet transformation makes it possible to represent an event window with a window width of 64 sampling steps (64-coefficient DWT) with fewer than 16 wavelet transformation coefficients and at the same time obtain sufficient information in respect of the signal, to guarantee reliable event classification in the probabilistic neural network 5.

For carrying out the wavelet transformation operation the transformation unit 3 includes a wavelet store 6 in which the reference wavelets are stored and a computing unit 4 which is connected to the adjusting/standardizing stage 28 for receiving the event signal standardized and centered in the window and to the wavelet store 6 for receiving the reference wavelets. Calculation of the coefficients, that is to say the actual wavelet transformation operation, takes place in the computing unit 4.

There are a number of calculation methods which are suitable for calculation of wavelet transformation. Equally there are a large number of suitable reference wavelets. For calculation of wavelet transformation in the computing unit 4, it is possible to select the set of reference wavelets used, for example having regard to the computing power which can be achieved. When selecting the calculation method and the reference wavelets however care is preferably to be taken to ensure that, when calculating wavelet transformation in the computing unit 4, the same calculation method and the same set of reference wavelets are used as are employed when calculating the comparative coefficients (see hereinafter).

The computing unit 4 outputs the result of wavelet transformation, that is to say the wavelet transformation coefficients, as a set of coefficients, to the probabilistic neural network 5 (abbreviated hereinafter to PNN).

The PNN 5 includes a PNN structure 8, an input layer 7 and an output layer or summation unit 9. The PNN structure 8 has a number of inner nodes and is connected to the input layer 7 which has a number of input nodes and to the summation unit 9 or output layer which has a number of output nodes.

The inner nodes of the PNN structure 8 each contain a given coefficient vector which contains comparative coefficients as a set of comparative values, a given comparative time interval and a given comparative standardization factor, and characterizes a given class of events. In the preferred embodiment the PNN structure 8 includes for each class just one node, but it is also possible to associate with a class a plurality of inner nodes with respective slightly different coefficient vectors so that a class is represented by a node cluster. The coefficient vectors are usually previously extracted from a plurality of signals of the signal shape which is typical for the event class.

The purpose of the input layer 7 of the PNN 5 is to receive the coefficients from the transformation unit 3 and the time interval and the standardization factor of the present IEGM from the adjusting/standardizing stage 28 and to distribute them uniformly over the inner nodes of the PNN structure 8.

In the inner nodes the respective coefficient vectors are compared to a signal vector which is formed from the coefficients received from the transformation unit 3, as well as the time interval and the standardization factor of the present IEGM, by forming the difference of the signal vector and the coefficient vector. In addition, probability values are associated with the ascertained vector differences, in which respect the probability value is greater in proportion to a decreasing vector difference. The operation of determining the probability values can include a Gaussian transfer function with selectable standard deviation sigma (which specifies the spacing of the points of inflexion of the curve from the center of the curve). The selectable standard deviation makes it possible to establish the limits, that is to say the maximum admissible deviation from the respective standard signal shape of a class.

The PNN structure 8 is connected to the summation unit 9 for transmitting the signal vector and the probability values ascertained for the signal vector.

The summation unit 9 has precisely one output node for each event class, for the recognition of which the apparatus according to the invention is designed. The output node receives the probability value of the signal vector, which is ascertained for the respective event class. If a plurality of inner nodes are associated with an event class, the corresponding output node receives all probability values of those inner nodes and calculates the average value of the corresponding probability values. In both cases the probability value of an output node of the summation unit 9 represents the probability of the IEGM or the triggering event belonging to the class represented by the output node. The event triggering the IEGM is associated with that class which involves the highest probability value in the summation unit 9, insofar as that probability value exceeds a classification threshold. If it does not exceed the classification threshold the event is classified as unknown and possibly used to trigger adaptation of the PNN structure, which results in recognition of a new event class. Finally the summation unit 9 outputs the signal vector and the event class with which it has been associated as the result of the classification procedure. The signal vector and the event class with which it has been associated is also transmitted to the updating unit 10 by the summation unit 9.

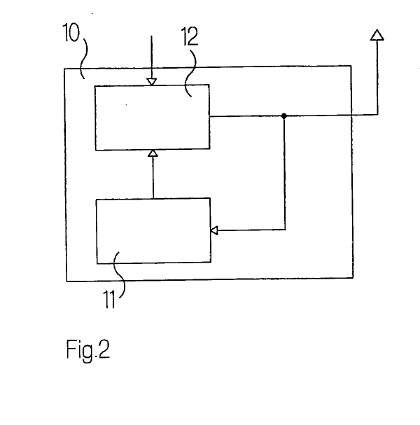

The updating unit 10 (see FIG. 2) includes a store 11 in which the coefficient vector which is the current one (that is to say which has been used hitherto) is stored for each inner node of the PNN structure 8. In addition it includes a combination unit 12 which is connected to the PNN 5 for receiving the signal vector and the event class with which it has been associated and to the store 11 for receiving the corresponding current coefficient vector.

The combination unit 12 provides for performing the operation of determining the new, that is to say updated, coefficient vector of the inner node, on the basis of the received signal vector and the current coefficient vector stored in the store 11. For that purpose the procedure involves multiplication of the coefficients contained in the signal vector by a factor a and multiplication of the coefficients contained in the current coefficient vector by a factor (1-α). The procedure then involves ascertaining the new (that is to say updated) coefficient vector on the basis of the two vectors multiplied in that way. In that case each new (updated) coefficient represents the sum of the corresponding coefficient of the current coefficient vector, multiplied by the factor (1-α) and the corresponding coefficient of the signal vector, multiplied by the factor α.

If the signal vector and the coefficient vector each contain for example 16 coefficients, the first new coefficient of the new coefficient vector is given by the sum of the first coefficient of the signal vector/ multiplied by the factor α, and the first coefficient of the current coefficient vector, multiplied by the factor (1-α), the second new coefficient of the new coefficient vector is given by the sum of the second coefficient of the signal vector, multiplied by the factor α, and the second coefficient of the current coefficient vector, multiplied by the factor (1-α), and so forth.

The described procedure provides that, in each updated coefficient vector, the coefficients of the signal vectors associated previously with the corresponding inner node, are taken into consideration, with an exponential weighting. In order adequately to take account of the physiological aspects involved, the factor a should be markedly less than 1. The update coefficient vector is finally transmitted to the PNN structure 8 and to the store 11 where it replaces the previous coefficient vector of the corresponding node.

As an alternative to exponential weighting of the coefficients from the signal vectors previously ascertained for the event class, it is also possible to form an average value. Each new coefficient of the new coefficient vector then represents an average value from the corresponding coefficients of a number of signal vectors previously associated with the inner node. The number can either be fixedly predetermined or it can include all signal vectors which within a predetermined, physiologically meaningful period of time, have been associated with the corresponding event class, that is to say the corresponding inner node. Then, to store the signal vectors, the updating unit 10 includes a further store which for example can also be in the form of a storage portion of the store 11.

The described operation of calculating the updated coefficient vector of an inner node and the replacement of the previous coefficient vector of that inner node are implemented in the present embodiment with that signal vector which is associated with the node. Alternatively however calculation and/or replacement can also take place only after each n-th, for example after each 10-th, signal vector which is associated with the inner node.

Although in the present embodiment the updating unit is arranged outside the classification unit 1, it can also be integrated into the classification unit 1, for example into the summation unit 9.

The present invention can be implemented both in the form of hardware and also in the form of software.

1. An apparatus for the classification of physiological events on the basis of physiological signals, said apparatus comprising:
a probabilistic neural network which is adapted to receive a set of values representing the physiological signal and which contains a number of event classes which represent physiological events and which are respectively determined by a number of comparative values, which network is adapted on the basis of the comparison of the set of values with the comparative values to implement an association of the physiological signal represented by the set of values with one of the event classes, and
an updating unit connected to the probabilistic neural network for updating the comparative values of an event class on the basis of the set of values of at least one physiological signal which has been associated with said event class in a preceding association operation.

2. The apparatus of claim 1, wherein:
the updating unit is so designed that upon updating of the comparative values an average value is formed from a number of value sets which have previously resulted in an association of the physiological signals which they represent with the event class to be updated and wherein the updating operation is effected on the basis of the average value formed in that way.

3. The apparatus of claim 1 wherein:
the updating unit is so designed that upon updating of the comparative values exponential weighting of a number of value sets which have previously resulted in an association of the physiological signals which they represent with the event class to be updated is effected and wherein the updating operation is effected on the basis of the exponentially weighted value sets.

4. The apparatus of claim 3, wherein: the updating unit is so designed that updating of an event class is effected after the association of a n-th value set with said event class, wherein that defines a predetermined number of value sets.

5. The apparatus of claim 4, wherein: different values for n are to be associated with different event classes.

6. The apparatus of claim 5, further comprising:
a signal input for the input of a physiological signal; and
a transformation unit which is connected to the signal input for receiving the physiological signal and which is adapted to implement a transformation of the physiological signal in such a way that as the output signal it outputs a number of values representing the physiological signal and based on the transformation operation; wherein the probabilistic neural network is connected to the transformation unit for receiving the values as the value set.

7. The apparatus of claim 6, wherein: the transformation unit is adapted for executing the transformation operation on the basis of wavelets and a transformation rule determining the values to be outputted using the wavelets.

8. An implantable medical device, comprising:
an apparatus for the classification of physiological events on the basis of physiological signals comprising:
a probabilistic neural network which is adapted to receive a set of values representing the physiological signal and which contains a number of event classes which represent physiological events and which are respectively determined by a number of comparative values, which network is adapted on the basis of the comparison of the set of values with the comparative values to implement an association of the physiological signal represented by the set of values with one of the event classes, and
an updating unit connected to the probabilistic neural network for updating the comparative values of an event class on the basis of the set of values of at least one physiological signal which has been associated with said event class in a preceding association operation.

9. The implantable medical advice of claim 8, wherein: the medical device is in the form of a cardiac pacemaker or defibrillator.

10. The apparatus of claim 1, wherein:
the updating unit is so designed that updating of an event class is effected after the association of a n-th value set with said event class, wherein that defines a predetermined number of value sets.

11. The apparatus of claim 2, wherein:
the updating unit is so designed that updating of an event class is effected after the association of a n-th value set with said event class, wherein that defines a predetermined number of value sets.

12. The apparatus of claim 10, wherein:
different values for n are to be associated with different event classes.

13. The apparatus of claim 11, wherein:
different values for n are to be associated with different event classes.

14. The apparatus of claim 1, further comprising:
a signal input for the input of a physiological signal; and
a transformation unit which is connected to the signal input for receiving the physiological signal and which is adapted to implement a transformation of the physiological signal in such a way that as the output signal it outputs a number of values representing the physiological signal and based on the transformation operation; wherein the probabilistic neural network is connected to the transformation unit for receiving the values as the value set.

15. The apparatus of claim 14, wherein:
the transformation unit is adapted for executing the transformation operation on the basis of wavelets and a transformation rule determining the values to be outputted using the wavelets.
